(19) United States
(12) Patent Application Publication
(10) Pub. No.: US 2026/0196045 A1
(43) Pub. Date: Jul. 9, 2026

(54) MEDICAL IMAGE PROCESSING APPARATUS, MEDICAL IMAGE PROCESSING METHOD, AND COMPUTER-READABLE NON-VOLATILE STORAGE MEDIUM STORING MEDICAL IMAGE PROCESSING PROGRAM

(71) Applicant: Canon Kabushiki Kaisha, Tokyo (JP)

(72) Inventors: Asateru KIMURA, Yokohama (JP); Yuki MATSUMOTO, Utsunomiya (JP); Yasuhito NEMOTO, Otawara (JP); Narumi KIKUCHI, Nasushiobara (JP)

(73) Assignee: Canon Kabushiki Kaisha, Tokyo (JP)

(21) Appl. No.: 19/382,378

(22) Filed: Nov. 7, 2025

(30) Foreign Application Priority Data
Jan. 7, 2025 (JP) 2025-002120

(51) Int. Cl. G06V 20/40; G06F 40/289; G16H 70/20; G06V 10/70

(52) U.S. Cl. CPC G06V 20/47; G06F 40/289; G06V 20/41; G16H 70/20; G06V 10/70; G06V 2201/034

(57) ABSTRACT

A medical image processing apparatus according to an embodiment includes a processing circuitry. Based on moving image data obtained by capturing a series of first procedures performed by a specific operator on a disease of a subject, the processing circuitry generates a first text corresponding to each of the first procedures. Based on sentence data in which information regarding a series of second procedures related to the disease is described, or based on moving image data obtained by capturing the second procedures, the processing circuitry generates a second text corresponding to each of the second procedures. The processing circuitry extracts a difference between each of the first procedure and each of the second procedure from the first text and the second text. The processing circuitry outputs information regarding the difference.

### CROSS-REFERENCE TO RELATED APPLICATION

This application is based upon and claims the benefit of priority from Japanese Patent Application No. 2025-002120, filed on January 7, 2025, the entire contents of which are incorporated herein by reference.

### FIELD

Embodiments described herein relate generally to a medical image processing apparatus, a medical image processing method, and a computer-readable non-volatile storage medium storing a medical image processing program.

### BACKGROUND

Conventionally, a standard technique of surgery (hereinbelow referred to as a standard procedure) is illustrated in textbooks, surgical guidelines, and the like. Therefore, the standard procedure can be passed on by textbooks, surgical guidelines, and the like. On the other hand, implicit knowledge (hereinbelow referred to as an expert procedure) about a procedure performed by a surgery expert (skilled person or specialist) is not sufficiently converted into a text, and may be difficult to pass on to other doctors. Therefore, for example, it may be difficult to improve the training efficiency of a beginner surgeon or the like.

### BRIEF DESCRIPTION OF THE DRAWING

FIG. 1 is a block diagram illustrating an example of a configuration of a medical information processing system including a medical information processing apparatus according to an embodiment;

FIG. 2 is a diagram illustrating an example of an outline in which a first text is generated by inputting moving image data of surgery and a first prompt into an LLM according to the embodiment;

FIG. 3 is a diagram illustrating an example of an outline in which a second text is generated by inputting a guideline of surgery and a second prompt into the LLM according to the embodiment;

FIG. 4 is a diagram illustrating an example of a similarity between character strings of purposes in the first text and the second text according to the embodiment;

FIG. 5 is a diagram illustrating an example of the first text and the second text associated with each other according to the purposes illustrated in FIG. 4 according to the embodiment;

FIG. 6 is a diagram illustrating an example of an outline for extracting a difference between an operation content in the first text and an operation content in the second text regarding the purposes in the first text and the second text according to the embodiment;

FIG. 7 is a diagram illustrating an example of an outline for extracting a difference between a name of an instrument in the first text and a name of an instrument in the second text regarding the purposes in the first text and the second text according to the embodiment;

FIG. 8 is a diagram illustrating an example of display of information regarding a difference extracted by a difference extraction function according to the embodiment;

FIG. 9 is a diagram illustrating an example of display of information regarding a difference extracted by the difference extraction function according to the embodiment;

FIG. 10 is a diagram illustrating an example of display of information regarding a difference extracted by the difference extraction function according to the embodiment;

FIG. 11 is a flowchart illustrating an example of a procedure of a conversion-into-text and difference display processing according to the embodiment; and

FIG. 12 is a diagram illustrating an example of an application case in the present application example of the embodiment.

### DETAILED DESCRIPTION

A medical image processing apparatus according to an embodiment includes a processing circuitry. Based on moving image data obtained by capturing a series of first procedures performed by a specific operator on a disease of a subject, the processing circuitry generates a first text corresponding to each of the first procedures. Based on sentence data in which information regarding a series of second procedures related to the disease is described, or based on moving image data obtained by capturing the second procedures, the processing circuitry generates a second text corresponding to each of the second procedures. The processing circuitry extracts a difference between each of the first procedure and each of the second procedure from the first text and the second text. The processing circuitry outputs information regarding the difference.

Hereinbelow, embodiments of a medical information processing apparatus, a medical information processing method, and a medical information processing program will be described with reference to the drawings. In the following embodiment, portions denoted by the same reference numerals perform similar operations, and redundant description will be omitted as appropriate. In addition, the medical information processing apparatus, the medical information processing method, and the medical information processing program may be referred to as a medical image processing apparatus, a medical image processing method, and a medical image processing program, respectively.

### Embodiment

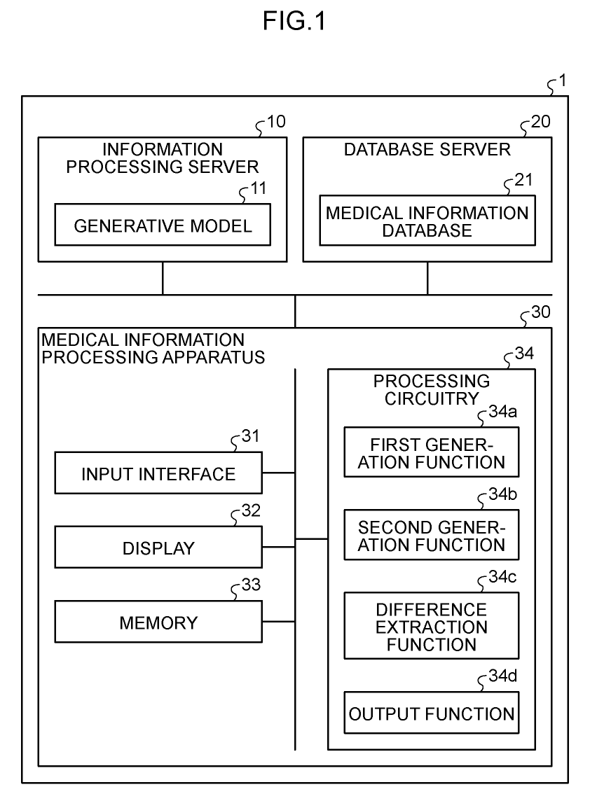

FIG. 1 is a block diagram illustrating an example of a configuration of a medical information processing system 1 including a medical information processing apparatus 30 according to an embodiment. Note that the medical information processing system 1 may be referred to as a medical image processing system. As illustrated in FIG. 1, the medical information processing system 1 according to the embodiment includes an information processing server 10, a database server 20, and a medical information processing apparatus 30. Note that the information processing server 10 and the database server 20 may be referred to as a medical image processing server and a medical database server, respectively. The information processing server 10 and the database server 20 are connected to the medical information processing apparatus 30 via a network such as the Internet and/or an intranet. Note that the connection destination with the medical information processing apparatus 30 is not limited to the information processing server 10 and the database server 20 described above, and another server and/or another database or the like may be connected.

The information processing server 10 includes a memory that stores a generative model 11 and a processor that executes the generative model 11. The generative model 11 is, for example, a large language model (LLM) or a multimodal model (MMM). Here, an example in which the generative model 11 is the LLM will be described. Note that the LLM may be replaced with a model such as the MMM. The generative model 11 is an AI model pre-trained using a large-scale corpus in the field of natural language processing, and has a function of, for example, when a question or instruction content is input as a sentence (prompt), generating and outputting a response sentence according to the sentence meaning of the input sentence.

The processor in the information processing server 10 reads the generative model 11 from the memory, and inputs a prompt output from the medical information processing apparatus 30 and data to be processed into the generative model 11, thereby generating a response sentence. The processor transmits the generated response sentence (output from the generative model 11) to the medical information processing apparatus 30. The generated response sentence, i.e., the answer from the generative model 11 corresponding to the prompt, includes medical information about a user. Note that the generative model 11 may be built in the medical information processing apparatus 30.

The database server 20 has a medical information database 21, that is, a memory that stores various types of medical information. The medical information database 21 stores, for example, moving image data obtained by capturing a series of a plurality of first procedures performed by a specific operator for a disease of a subject. The specific operator is, for example, a surgery expert. The first procedure may be referred to as an expert procedure. To the moving image data are attached information regarding the specific operator, information regarding a disease (for example, the name of the disease and the state of the disease), and the like. At this time, the specific operator is registered in the medical information database 21 as an expert regarding surgery for the disease.

Furthermore, the database server 20 stores sentence data regarding normal surgery (standard surgery) such as a guideline regarding implementation of surgery on a subject and a textbook regarding the surgery. That is, the sentence data includes a guideline regarding implementation of the surgery. A procedure regarding the standard surgery is hereinbelow referred to as a second procedure. The second procedure may be referred to as a normal procedure (standard procedure). In addition, to the sentence data, information regarding a disease is attached. Note that the database server 20 may have various types of medical information such as a medical paper, and a column and a report in a medical journal.

In addition, the medical information database 21 may include not only various types of medical information but also information such as an inspection, a diagnosis, a treatment, and handling. In addition, in a case where the medical information database 21 is stored in a hospital information system (hereinbelow referred to as an HIS), the medical information database 21 includes various types of medical information stored in the HIS. At this time, the database server 20 is achieved by an HIS server.

The medical information processing apparatus 30 acquires moving image data from the database server 20 via the network and stores the moving image data in a memory 33. In addition, the medical information processing apparatus 30 transmits a prompt and the moving image data to the information processing server 10 via the network. The medical information processing apparatus 30 acquires a response sentence output from the generative model 11 from the information processing server 10 via the network.

In addition, the medical information processing apparatus 30 accesses the database server 20 via the network and searches the medical information database 21. For example, the medical information processing apparatus 30 searches, using the name of a disease attached to the moving image data as a search condition, for sentence data regarding the disease that matches the name of the disease. Note that the search condition in the search for the sentence data is not limited to the information attached to the moving image data, and for example, information of an electronic medical record regarding the moving image data may be used as the search condition. The search in the medical information database 21 may be executed by a processor in the database server 20 according to the search condition output from the medical information processing apparatus 30, for example. The medical information processing apparatus 30 acquires a search result from the database server 20.

The medical information processing apparatus 30 is achieved by, for example, various terminals used by a user. The various terminals are, for example, a personal computer and a tablet terminal that can be connected to a network. Note that the medical information processing apparatus 30 may be achieved by a terminal apparatus in a hospital. As illustrated in FIG. 1, the medical information processing apparatus 30 includes an input interface 31, a display 32, the memory 33, and a processing circuitry 34.

The input interface 31 is achieved by a trackball, a switch, a button, a mouse, a keyboard, a touch pad that receives an input operation when an operation surface is touched, a touch screen in which a display screen and the touch pad are integrated, a non-contact input circuitry using an optical sensor, a voice input circuitry (such as a microphone), and the like used to perform various instructions, various settings, and the like. The input interface 31 converts an input operation received from the user into an electric signal and outputs the electric signal to the processing circuitry 34. For example, the input interface 31 receives input of an instruction for selecting moving image data to be input into the generative model 11 according to an instruction of the user.

Note that the input interface 31 is not limited to one including a physical operation component such as a mouse and a keyboard. For example, an electric signal processing circuitry that receives an electric signal corresponding to an input operation from an external input apparatus provided separately from the medical information processing apparatus 30 and outputs the electric signal to the processing circuitry 34 is also included as an example of the input interface 31. The input interface 31 is an example of an input unit and may be referred to as an operation unit.

The display 32 displays various types of information under the control of an output function 34d. For example, the display 32 displays a graphical user interface (GUI) used to receive an instruction of the user, a prompt to the generative model 11, various display screens based on an answer from the generative model 11, and the like. The display screen will be described below. The display 32 is, for example, a liquid crystal display or a cathode ray tube (CRT) display. The display 32 is an example of a display unit.

The memory 33 is achieved by, for example, a random access memory (RAM), a semiconductor memory element such as a flash memory, a hard disk, or an optical disk. For example, the memory 33 stores various types of data generated by various types of processing by the processing circuitry 34 described below. In addition, the memory 33 stores a response sentence (answer) output from the generative model 11. In addition, the memory 33 stores a search result obtained from the medical information database 21. In addition, the memory 33 stores a program for each circuitry included in the medical information processing apparatus 30 to fulfill its function. The memory 33 is an example of a storage unit.

The processing circuitry 34 controls the entire operation of the medical information processing apparatus 30 by executing a first generation function 34a, a second generation function 34b, a difference extraction function 34c, and an output function 34d. The processing circuitry 34 that fulfills the first generation function 34a corresponds to a first generation unit. The processing circuitry 34 that fulfills the second generation function 34b corresponds to a second generation unit. The processing circuitry 34 that fulfills the difference extraction function 34c corresponds to a difference extraction unit. The processing circuitry 34 that fulfills the output function 34d corresponds to an output unit.

The processing circuitry 34 reads a program corresponding to the first generation function 34a from the memory 33 and executes the program. By doing so, based on moving image data obtained by capturing a series of a plurality of first procedures performed by a specific operator on a disease of a subject, the first generation function 34a generates a plurality of first texts associated with the plurality of first procedures. Specifically, the first generation function 34a reads, from the database server 20 or the memory 33, the moving image data obtained by capturing a series of first procedures performed by the specific operator on the disease of the subject. Subsequently, the first generation function 34a inputs the moving image data and a first prompt into the generative model 11 in the information processing server 10.

Note that the moving image data may include a series of a plurality of first procedures performed by the specific operator. At this time, the first generation function 34a generates the plurality of first texts associated with the plurality of first procedures.

The first prompt is, for example, "This is a moving image of surgery to treat XXX. From the moving image of surgery, convert a purpose of a procedure, a content of the procedure, and an instrument to be used into texts.". In addition, the purpose of the procedure may be simply referred to as a procedure. In XXX in the first prompt, the name of the disease attached to the moving image data is set. The first generation function 34a acquires an output from the generative model 11 from the information processing server 10. The first generation function 34a generates the first text associated with the first procedure in the moving image data on the basis of the output from the generative model 11. For example, the first generation function 34a generates the plurality of first texts associated with the plurality of first procedures in the moving image data on the basis of the output from the generative model 11. The first generation function 34a stores the first text in the memory 33.

Note that the first prompt may further include an instruction for specifying a frame number associated with the purpose of the procedure in the moving image data. At this time, the first generation function 34a generates the plurality of first texts associated with the plurality of first procedures in the moving image data and the frame numbers on the basis of the output from the generative model 11. That is, the generative model 11 outputs the frame number of the moving image related to the purpose in addition to the output related to the first text. At this time, the first generation function 34a stores the first text and the frame number in the memory 33 in association with the purpose in the first text.

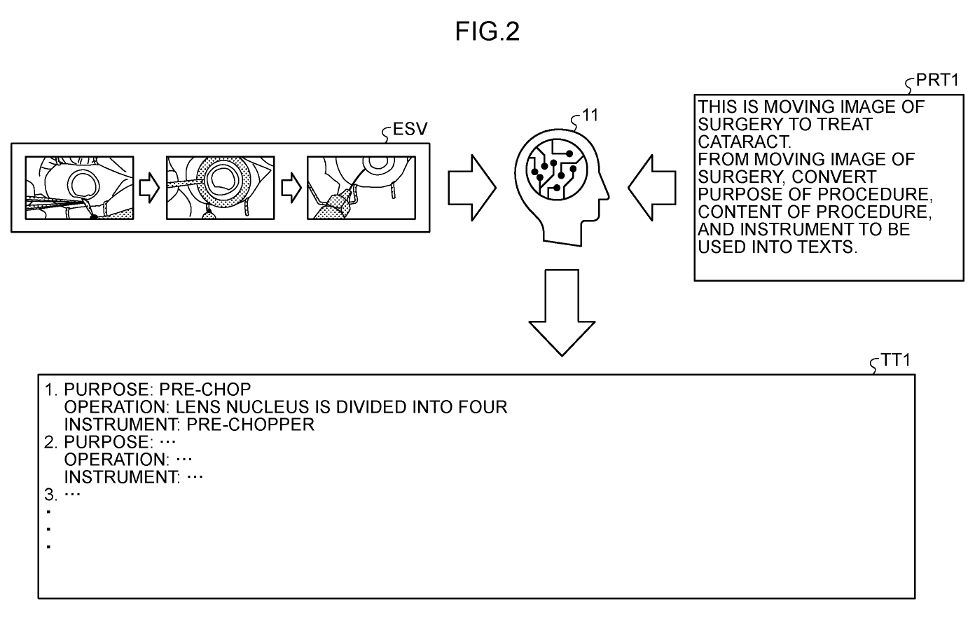

FIG. 2 is a diagram illustrating an example of an outline in which a first text TT1 is generated by inputting moving image data ESV of surgery related to cataract and a first prompt PRT1 into the generative model 11. As illustrated in FIG. 2, for example, a deep machine learning model for moving image recognition such as GPT4-o is used as the generative model 11. As illustrated in FIG. 2, the first text TT1 includes a text for each of items including the purpose of the procedure, the operation content of the procedure, and the name of the instrument used in the procedure. The text in the item is generated for each first procedure. As illustrated in FIG. 2, the first text TT1 corresponds to structured data of texts for the moving image data.

The processing circuitry 34 reads a program corresponding to the second generation function 34b from the memory 33 and executes the program. By doing so, based on sentence data in which information regarding a series of second procedures, which are normal, related to the disease in the moving image data ESV is described, the second generation function 34b generates a second text associated with the second procedure.

Note that the sentence data may include information regarding a series of a plurality of second procedures, which are normal. At this time, the second generation function 34b generates a plurality of second texts associated with the plurality of second procedures on the basis of the sentence data.

Specifically, the second generation function 34b specifies, for example, the name of the disease in the moving image data ESV. Subsequently, the second generation function 34b searches the medical information database 21 in the database server 20, using the name of the disease as a search condition. By the search, the second generation function 34b acquires the sentence data corresponding to the name of the disease related to the moving image data from the medical information database 21. The acquired sentence data is data having the name of the disease corresponding to that of the moving image data.

Subsequently, the second generation function 34b inputs the sentence data and a second prompt into the generative model 11 in the information processing server 10. Hereinbelow, for convenience of description, the second prompt used in the second generation function 34b is, for example, "The following sentences are texts related to XXX. From the sentences, convert a purpose of a procedure, a content of the procedure, and an instrument to be used into texts.". In addition, the purpose of the procedure may be simply referred to as a procedure. In XXX in the second prompt, the name of the disease attached to the moving image data is set.

The second generation function 34b acquires an output from the generative model 11 from the information processing server 10. The second generation function 34b generates the plurality of second texts associated with the plurality of second procedures in the sentence data on the basis of the output from the generative model 11. The second generation function 34b stores the second text in the memory 33.

Note that the second prompt may further include an instruction for specifying sentence data (for example, a character string of a guideline) associated with the purpose of the procedure in the second text. At this time, the second generation function 34b generates the plurality of second texts associated with the plurality of second procedures and the character strings of the guideline in the sentence data on the basis of the output from the generative model 11.

That is, the generative model 11 outputs the character string of the guideline related to the purpose in addition to the output related to the second text. At this time, the second generation function 34b stores the second text and the character string of the guideline in the memory 33 in association with the purpose in the second text.

Note that, in the above description, the generative model used by the first generation function 34a and the generative model used by the second generation function 34b are the same generative model 11, but the present embodiment is not limited thereto, and the generative models may be different. For example, the generative model used by the first generation function 34a may be an LLM intended for moving image recognition, and the generative model used by the second generation function 34b may be an LLM intended for sentence data recognition.

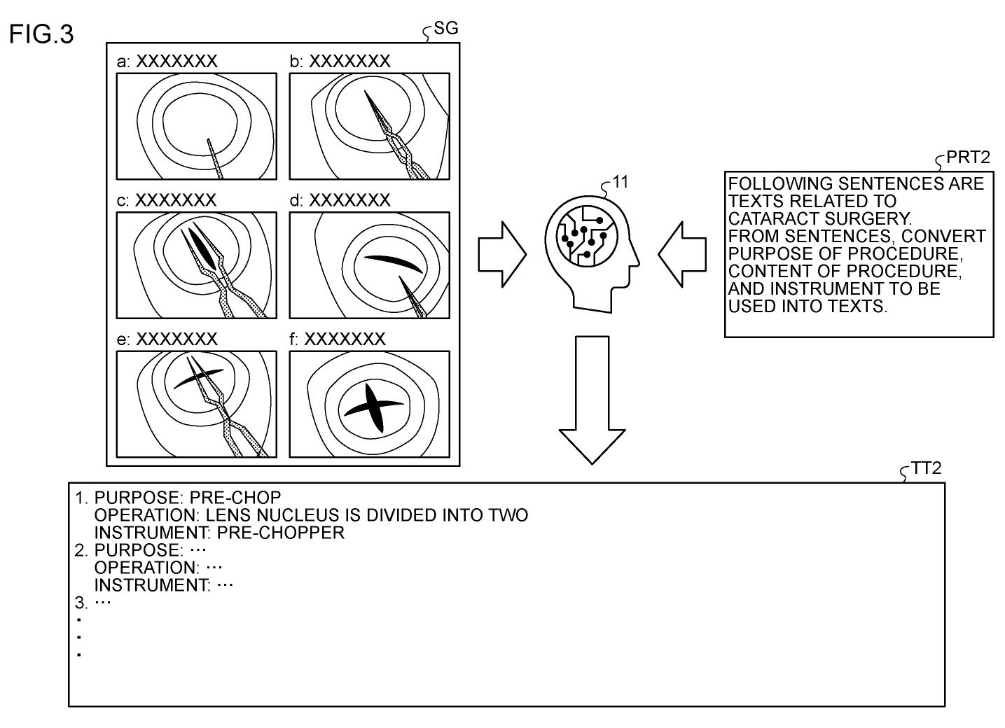

FIG. 3 is a diagram illustrating an example of an outline in which the second text TT2 is generated by inputting a guideline SG of surgery related to cataract and a second prompt PRT2 into the generative model 11. As illustrated in FIG. 3, as in FIG. 2, for example, a deep machine learning model for moving image recognition such as GPT4-o is used as the generative model 11. As illustrated in FIG. 3, the second text TT2 includes a text for each of items including the purpose of the procedure, the operation content of the procedure, and the name of the instrument used in the procedure. The text in the item is generated for each second procedure. As illustrated in FIG. 3, the second text TT2 corresponds to structured data of texts for the sentence data.

The processing circuitry 34 reads a program corresponding to the difference extraction function 34c from the memory 33 and executes the program. By doing so, the difference extraction function 34c extracts a difference between the first procedure and the second procedure from the first text TT1 and the second text TT2. For example, the difference extraction function 34c extracts the difference for each of the items.

Specifically, the difference extraction function 34c calculates a similarity between the texts indicating the purposes in the first text TT1 and the second text TT2. A known method using cosine similarity between two texts, edit distance, a BERT(Bidirectional Encoder Representations from Transformers) score (similarity between output vectors of BERT), and the like is applicable to the calculation of the similarity between the two texts, and thus, description thereof is omitted.

In the first text TT1 and the second text TT2, the difference extraction function 34c uses a similarity between the texts indicating the purposes to execute association according to the purposes. For example, the difference extraction function 34c associates with each other two purposes having the highest similarity and a higher similarity than a predetermined threshold in the first text TT1 and the second text TT2. The predetermined threshold is, for example, 0.8, which is set in advance and stored in the memory 33. Note that the predetermined threshold is not limited to 0.8, and may be another value.

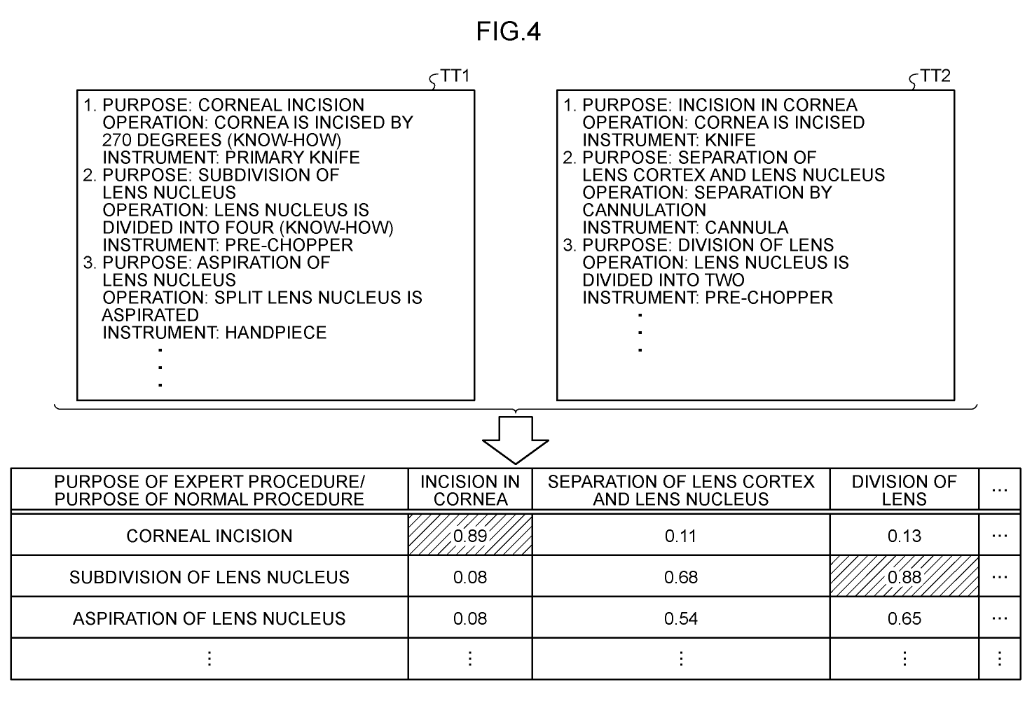

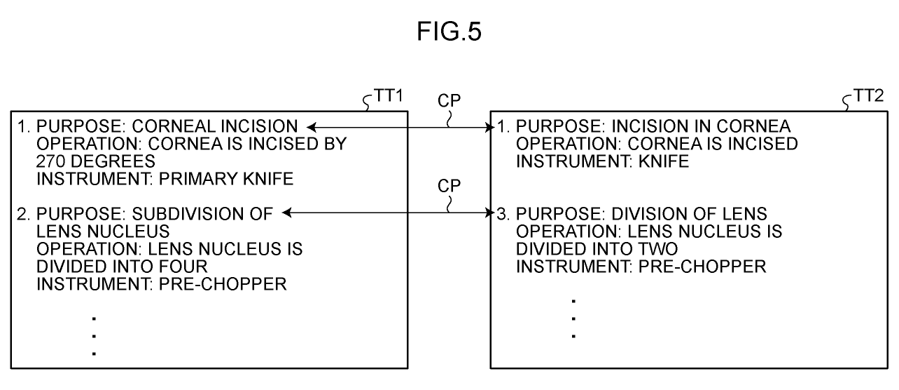

FIG. 4 is a diagram illustrating an example of a similarity between character strings of purposes in the first text TT1 and the second text TT2. FIG. 5 is a diagram illustrating an example of the first text TT1 and the second text TT2 associated with each other according to the purposes illustrated in FIG. 4. As illustrated in FIG. 4, in a case where the purpose of the expert procedure is "corneal incision" and the purpose of the normal procedure is "incision in cornea", the similarity is 0.89. Therefore, as illustrated as CP in FIG. 5, the difference extraction function 34c associates "corneal incision", which is the purpose of the expert procedure, with "incision in cornea", which is the purpose of the normal procedure.

In addition, as illustrated in FIG. 4, in a case where the purpose of the expert procedure is "subdivision of lens nucleus" and the purpose of the normal procedure is "division of lens", the similarity is 0.88. Therefore, as illustrated as CP in FIG. 5, the difference extraction function 34c associates "subdivision of lens nucleus", which is the purpose of the expert procedure, with "division of lens", which is the purpose of the normal procedure.

The difference extraction function 34c extracts a difference in each of a plurality of items related to the associated purposes in the first text TT1 and the second text TT2. Hereinbelow, in order to make the description specific, the description will be given on the assumption that the items targeted for extraction of the difference are the operation content and the name of the instrument used in the procedure. At this time, the difference extraction function 34c extracts a difference in the text of the operation content and the name of the instrument related to the associated purposes in the first text TT1 and the second text TT2.

Specifically, regarding the associated purposes, the difference extraction function 34c inputs a text (character string) of the operation content in the first text TT1 and a text (character string) of the operation content in the second text TT2 into a sentence difference detection model. Since a known pre-trained model can be applied as the sentence difference detection model, the description thereof will be omitted. The sentence difference detection model is stored in the memory 33. The sentence difference detection model extracts a difference between the text (character string) of the operation content in the first text TT1 and the text (character string) of the operation content in the second text TT2.

Further, regarding the associated purposes, the difference extraction function 34c inputs a text (character string) of the instrument in the first text TT1 and a text (character string) of the instrument in the second text TT2 into the sentence difference detection model. The sentence difference detection model extracts a difference between the text (character string) of the instrument in the first text TT1 and the text (character string) of the instrument in the second text TT2.

By doing so, the difference extraction function 34c specifies a word serving as a determination basis of detection and output of the difference by the sentence difference detection model. The word is specified by, for example, weight visualization of attention in BERT. The difference extraction function 34c stores the extracted difference in the memory 33.

Note that the sentence difference detection model may be built in the information processing server 10. At this time, the difference extraction function 34c transmits the texts (character strings) of the operation contents and the instruments in the first text TT1 and the second text TT2 related to the associated purposes to the information processing server 10, and receives the extracted difference.

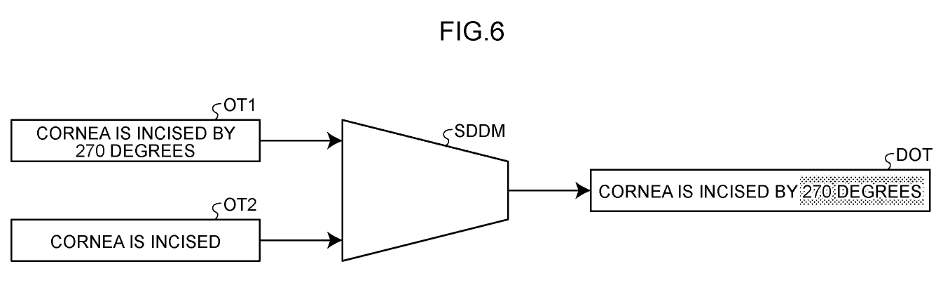

FIG. 6 is a diagram illustrating an example of an outline for extracting a difference between the operation content in the first text TT1 and the operation content in the second text TT2 regarding the purposes in the first text TT1 and the second text TT2. As illustrated in FIG. 6, the purpose in the first text TT1 is corneal incision, and the purpose in the second text TT2 is incision in cornea. An operation content OT1 in the first text TT1 is the cornea is incised by 270 degrees, and an operation content OT2 in the second text TT2 is the cornea is incised. As illustrated in FIG. 6, a difference (hereinbelow referred to as an operation difference) DOT between the operation content OT1 in the first text TT1 and the operation content OT2 in the second text TT2 is extracted by a sentence difference detection model SDDM.

As illustrated in FIG. 6, the operation content (the cornea is incised by 270 degrees) OT1 in the first text TT1 and the operation content (the cornea is incised) OT2 in the second text TT2 are input into the sentence difference detection model SDDM. As a result, the sentence difference detection model SDDM outputs the operation difference DOT. At this time, as illustrated in FIG. 6, in the operation difference DOT, the word (270 degrees) serving as the basis for determination of the difference by the sentence difference detection model SDDM is indicated by shading.

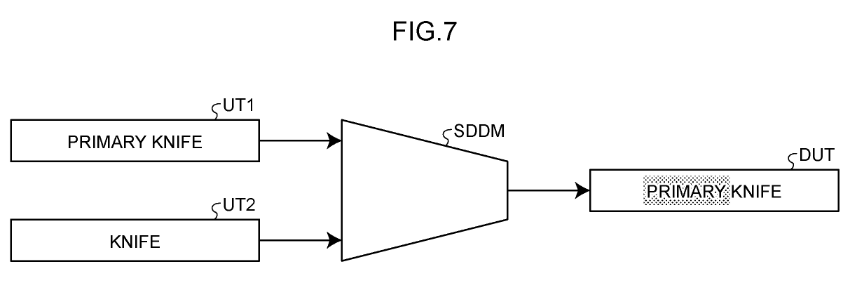

FIG. 7 is a diagram illustrating an example of an outline for extracting a difference between the name of the instrument in the first text TT1 and the name of the instrument in the second text TT2 regarding the purposes in the first text TT1 and the second text TT2. As illustrated in FIG. 7, the purpose in the first text TT1 is corneal incision, and the purpose in the second text TT2 is incision in cornea. As illustrated in FIG. 7, a difference (hereinbelow referred to as an instrument difference) DUT between the name of the instrument (primary knife) UT1 in the first text TT1 and the name of the instrument (knife) UT2 in the second text TT2 is extracted by the sentence difference detection model SDDM.

As illustrated in FIG. 7, the name of the instrument (primary knife) UT1 in the first text TT1 and the instrument name (knife) UT2 in the second text TT2 are input into the sentence difference detection model SDDM. As a result, the sentence difference detection model SDDM outputs the instrument difference DUT. At this time, as illustrated in FIG. 7, in the instrument difference DUT, the word (primary) serving as the basis for determination of the difference by the sentence difference detection model SDDM is indicated by shading.

The processing circuitry 34 reads a program corresponding to the output function 34d from the memory 33 and executes the program. By doing so, the output function 34c outputs information regarding the difference extracted by the difference extraction function 34c. For example, the output function 34d emphasizes the information regarding the difference and displays the information on the display 32.

Specifically, the output function 34d identifies the difference by color using a blue marker in the normal procedure and a red marker for know-how in the expert procedure, highlights, or the like, to display the difference on the display 32 in an emphasized manner. Furthermore, the output function 34d displays on the display 32 information regarding the first procedure (expert procedure) not included in the sentence data as the information regarding the difference. In addition, the output function 34d displays on the display 32 so that the associated purposes in the first text TT1 and the second text TT2 can be visually recognized.

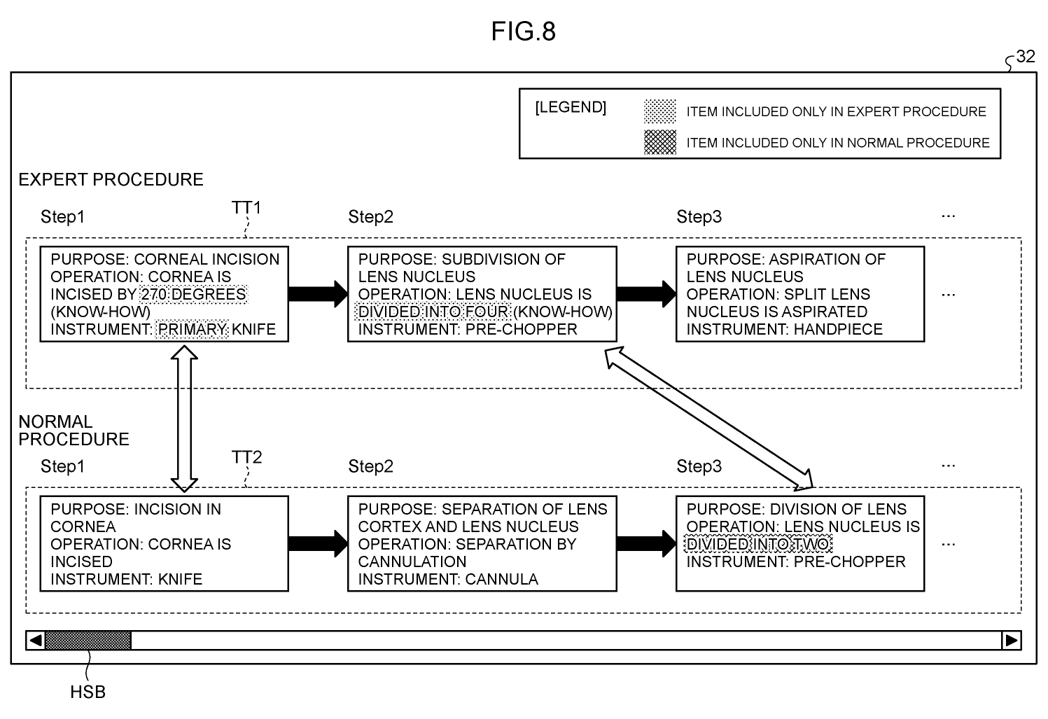

FIG. 8 is a diagram illustrating an example of display of information regarding the difference extracted by the difference extraction function 34c. As illustrated in FIG. 8, for the expert procedure (first procedure) and the normal procedure (second procedure), the first text TT1 and the second text TT2 are displayed by purpose in chronological order (step order) on the display 32.

In addition, the display 32 can display information regarding the difference for each predetermined item (purpose, operation, instrument, or the like) common to the expert procedure (first procedure) and the normal procedure (second procedure). For example, regarding a purpose (corneal incision, division of lens, or the like) common to the expert procedure (first procedure) and the normal procedure (second procedure), information regarding a difference in the purpose can be displayed on the display 32. Regarding an instrument (knife, pre-chopper, or the like) common to the expert procedure (first procedure) and the normal procedure (second procedure), information regarding a difference in the instrument can be displayed on the display 32.

Note that an item for information regarding a difference to be displayed on the display 32 may be designated. For example, by designating a purpose (corneal incision, division of lens, or the like) common to the expert procedure (first procedure) and the normal procedure (second procedure), information regarding a difference in the purpose can be displayed on the display 32. By designating an instrument (knife, pre-chopper, or the like) common to the expert procedure (first procedure) and the normal procedure (second procedure), information regarding a difference in the instrument can be displayed on the display 32.

At this time, as illustrated in FIG. 8, in the expert procedure (first procedure) and the normal procedure (second procedure), the output function 34d displays the difference on the display 32 with a different highlight (in FIG. 8, different shading). The first text TT1 and the second text TT2 are scrollable along a timeline (chronology, steps) according to an operation of a horizontal scroll bar HSB.

In addition, as illustrated in FIG. 8, regarding the associated purposes (procedures) in the expert procedure (first procedure) and the normal procedure (second procedure), the output function 34d displays on the display 32 the association (In FIG. 8, a double-headed arrow) between the steps of the procedures. That is, the output function 34d displays the association relationship between the purposes on the display 32 while maintaining the timeline of the expert procedure and the normal procedure.

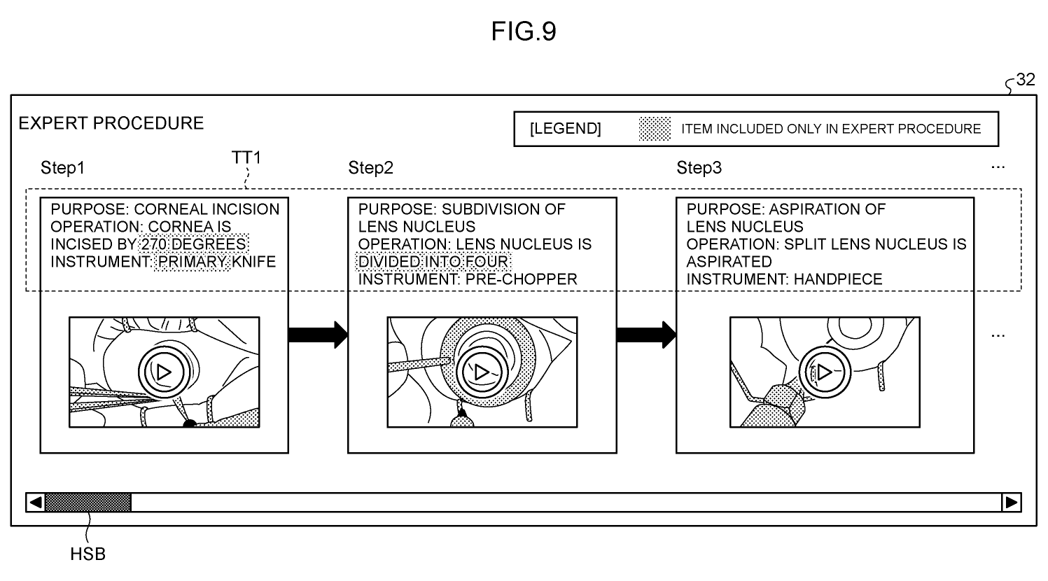

FIG. 9 is a diagram illustrating an example of display of information regarding the difference extracted by the difference extraction function 34c. As illustrated in FIG. 9, for the expert procedure (first procedure), the first text TT1 and the moving image data associated with each purpose are displayed by purpose in chronological order (step order) on the display 32. For example, the output function 34d displays on the display 32 the moving image data associated with the first procedure. Specifically, as illustrated in FIG. 9, in the expert procedure (first procedure), the output function 34d displays the difference with a highlight (in FIG. 9, shading) and the image with the smallest frame number out of the plurality of frame numbers associated with the purposes in the moving image data on the display 32.

At this time, when the image with the smallest frame number is selected, the output function 34d reproduces and displays the moving image data associated with the purpose by using the information of the frame number of the moving image data associated with the purpose. As illustrated in FIG. 9, the first text TT1 and the smallest frame number associated with the purpose are scrollable along the timeline (chronology, steps) according to the operation of the horizontal scroll bar HSB.

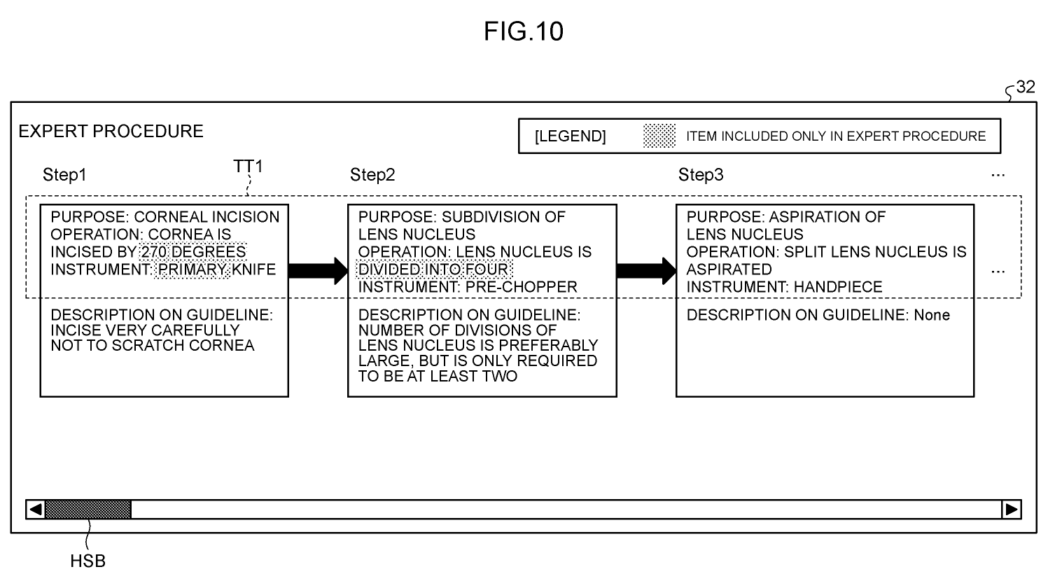

FIG. 10 is a diagram illustrating an example of display of information regarding the difference extracted by the difference extraction function 34c. As illustrated in FIG. 10, the output function 34d displays on the display 32 information (for example, a character string of a guideline) of the text of the normal procedure as auxiliary information for the user with respect to the text of the expert procedure. For example, by the output function 34d, the first text TT1 and the character string of the guideline associated with each purpose are displayed by purpose in chronological order (step order) on the display 32.

Specifically, as illustrated in FIG. 10, in the expert procedure (first procedure), the output function 34d displays the difference with a highlight (in FIG. 10, shading) and the description on the guideline associated with the purpose on the display 32. In addition, as illustrated in FIG. 10, the first text TT1 and the information of the text of the normal procedure associated with the purpose are scrollable along the timeline (chronology, steps) according to the operation of the horizontal scroll bar HSB.

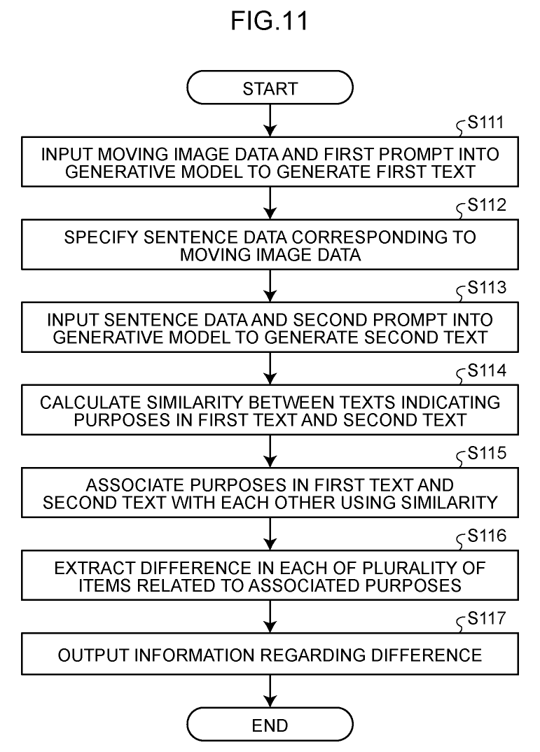

The overall configuration of the medical information processing system 1 according to the embodiment has been described above. Hereinbelow, processing (hereinbelow referred to as conversion-into-text and difference display processing) of converting the processes, the operations, and the instruments to be used, which are different in the normal procedure and the expert procedure, into texts and displaying the texts will be described. FIG. 11 is a flowchart illustrating an example of a procedure of the conversion-into-text and difference display processing.

### Conversion-into-text and Difference Display Processing

### Step S111

The processing circuitry 34 inputs moving image data obtained by capturing expert procedures by a specific operator and the first prompt PRT1 into the generative model 11 by the first generation function 34a. The first generation function 34a generates a plurality of first texts TT1 associated with a plurality of first procedures (expert procedures) on the basis of the output from the generative model 11. The first generation function 34a stores the plurality of generated first texts TT1 in the memory 33.

### Step S112

The processing circuitry 34 extracts, for example, the name of the disease attached to the moving image data from the moving image data by the second generation function 34b. The second generation function 34b searches the medical information database 21 using the name of the disease extracted as a search condition. The second generation function 34b specifies sentence data related to the same disease as the name of the disease related to the moving image data by the search. Note that the processing in this step may be executed before Step S111.

### Step S113

The processing circuitry 34 inputs the specified sentence data related to the moving image data and the second prompt PRT2 into the generative model 11 by the second generation function 34b. The second generation function 34b generates a plurality of second texts TT2 associated with a plurality of second procedures (normal procedures) on the basis of the output from the generative model 11. The second generation function 34b stores the plurality of generated second texts TT2 in the memory 33.

### Step S114

The processing circuitry 34 calculates a similarity between the texts indicating the purposes in the first text TT1 and the second text TT2 by a known method by the difference extraction function 34c. The difference extraction function 34c stores the calculated similarity in the memory 33.

### Step S115

The processing circuitry 34 associates the purposes in the first text TT1 and the second text TT2 with each other using a predetermined threshold and the similarity by the difference extraction function 34c. At this time, the difference extraction function 34c may associate the purposes with each other by integrating (combining) a plurality of purposes (for example, purposes in the procedures before and after the purpose of interest). That is, the difference extraction function 34c may use the similarity between the texts in which the purposes are combined to execute association according to the purposes. The difference extraction function 34c stores the associated purposes in the memory 33.

### Step S116

The processing circuitry 34 extracts a difference in each of a plurality of items related to the associated purposes in the first text TT1 and the second text TT2 by the difference extraction function 34c. The plurality of items include, for example, the operation content and the name of the instrument. Note that the plurality of items used for extraction of the difference is not limited to the operation content and the name of the instrument. For example, in the first text TT1 and the second text, in a case where execution time for each procedure is output by the generative model 11, the item used for extraction of the difference may be the execution time. Furthermore, in a case where the purposes are combined, the difference extraction function 34c may extract a difference in the text of the operation content and the name of the instrument related to the combined and associated purposes from the first text TT1 and the second text TT2.

### Step S117

The processing circuitry 34 outputs information regarding the difference by the output function 34d. For example, as illustrated in FIGS. 8 to 10, the output function 34d displays on the display 32 the information regarding the difference of the first text TT1 from the second text TT2.

The medical information processing apparatus 30 according to the present embodiment described above, based on moving image data obtained by capturing a series of first procedures performed by a specific operator on a disease of a subject, generates a first text associated with the first procedure, and based on sentence data in which information regarding a series of second procedures, which are normal, related to the disease is described, generates a second text associated with the second procedure. For example, the medical information processing apparatus 30 according to the present embodiment, based on moving image data ESV obtained by capturing a series of a plurality of first procedures performed by a specific operator on a disease of a subject, generates a plurality of first texts TT1 associated with the plurality of first procedures, and based on sentence data SG in which information regarding a series of a plurality of second procedures, which are normal, related to the disease is described, generates a plurality of second texts TT2 associated with the plurality of second procedures. Subsequently, the medical information processing apparatus 30 according to the present embodiment extracts a difference between the first procedure and the second procedure from the first text TT1 and the second text TT2, and outputs information regarding the difference.

Furthermore, in the medical information processing apparatus 30 according to the present embodiment, the specific operator is registered as an expert regarding surgery for the disease. Furthermore, in the medical information processing apparatus 30 according to the present embodiment, to the moving image data ESV, information regarding the specific operator is attached. Furthermore, in the medical information processing apparatus 30 according to the present embodiment, the sentence data SG includes a guideline regarding implementation of the surgery.

Furthermore, the medical information processing apparatus 30 according to the present embodiment inputs a first prompt PRT1 used to generate the first text TT1 from the moving image data ESV and the moving image data ESV into a pre-trained generative model 11 to generate the first text TT1. Furthermore, the medical information processing apparatus 30 according to the present embodiment inputs a second prompt PRT2 used to generate the second text TT2 from the sentence data SG and the sentence data SG into the pre-trained generative model 11 to generate the second text TT2.

Furthermore, in the medical information processing apparatus 30 according to the present embodiment, the first text TT1 and the second text TT2 each include a text for each of items including a purpose of a procedure, an operation content of the procedure, and a name of an instrument used in the procedure, and the medical information processing apparatus 30 extracts the difference between the first procedure and the second procedure for each of the items.

Furthermore, in the first text TT1 and the second text TT2, the medical information processing apparatus 30 according to the present embodiment uses a similarity between the texts indicating the purposes to execute association according to the purposes, and extracts the difference in the text of the operation content and the name of the instrument related to the associated purposes. Furthermore, in the first text TT1 and the second text TT2, the medical information processing apparatus 30 according to the present embodiment uses a similarity between the texts in which the purposes are combined to execute association according to the purposes, and extracts the difference in the text of the operation content and the name of the instrument related to the associated purposes.

Furthermore, the medical information processing apparatus 30 according to the present embodiment emphasizes the information regarding the difference extracted and displays the information on a display 32. Furthermore, the medical information processing apparatus 30 according to the present embodiment displays on the display 32 information regarding one first procedure not included in the sentence data as the information regarding the difference extracted. Furthermore, the medical information processing apparatus 30 according to the present embodiment displays on the display 32 so that the associated purposes in the first text TT1 and the second text TT2 can be visually recognized.

Therefore, according to the medical information processing apparatus 30 of the present embodiment, it is possible to distinguish the normal procedure from the expert procedure using the moving image of surgery by the expert and the guideline/textbook, convert the normal procedure and the expert procedure into texts, and specify and display the difference in the operation of the surgery and the instrument of the surgery between the first text TT1 regarding the expert procedure and the second text TT2 regarding the normal procedure. That is, according to the medical information processing apparatus 30 of the present embodiment, in a case where there is a difference between the first text TT1 of the expert procedure extracted from the moving image of the surgery and the second text TT2 of the normal procedure extracted from the textbook/guideline, it is considered that the difference is caused by the expert procedure and the normal procedure.

Therefore, according to the medical information processing apparatus 30 of the present embodiment, it is possible to convert the implicit knowledge about the procedure possessed by the expert into a text and easily provide the user with the implicit knowledge. Therefore, according to the medical information processing apparatus 30 of the present embodiment, it is possible to convert the process, the operation, and the instrument to be used of the expert procedure, which are different from those of the normal procedure, into texts, and easily pass on the implicit knowledge about the procedure possessed by the expert of the surgery to the user. Based on the above, according to the medical information processing apparatus 30 of the present embodiment, it is possible to efficiently train a medical worker such as a surgeon.

### Application Example

The present application example is to use moving image data of the normal procedure instead of the sentence data. Hereinbelow, in order to simplify the description, in the present application example, the moving image data ESV described in the embodiment will be hereinbelow referred to as first moving image data, and the moving image data of the normal procedure will be referred to as second moving image data. The second moving image data may be referred to as normal moving image data. In addition, in the description of the present application example, functions different from those of the embodiment will be described.

Based on the second moving image data obtained by capturing a series of a plurality of second procedures, which are normal, related to the disease in the first moving image data ESV, the processing circuitry 34 generates, by the second generation function 34b, a plurality of second texts associated with the plurality of second procedures. Specifically, the second generation function 34b specifies, for example, the name of the disease in the first moving image data ESV.

Subsequently, the second generation function 34b searches the medical information database 21 in the database server 20, using the name of the disease as a search condition. By the search, the second generation function 34b acquires second moving image data corresponding to the name of the disease related to the first moving image data ESV from the medical information database 21. The acquired second moving image data is moving image data having the name of the disease corresponding to that of the first moving image data ESV.

Subsequently, the second generation function 34b inputs the second moving image data and the first prompt PRT1 into the generative model 11 in the information processing server 10. The second generation function 34b acquires an output from the generative model 11 from the information processing server 10. The second generation function 34b generates a plurality of second texts TT2 associated with a plurality of second procedures in the second moving image data on the basis of the output from the generative model 11. The second generation function 34b stores the second texts TT2 in the memory 33.

The conversion-into-text and difference display processing in the present application example corresponds to that in which the moving image data is replaced with the first moving image data and the sentence data is replaced with the second moving image data in the flowchart in FIG. 11. In the present application example, the moving image data is replaced with the first moving image data in S111 and S112 in FIG. 11. In addition, in S113 of FIG. 11, the sentence data is replaced with the second moving image data. Therefore, description of the conversion-into-text and difference display processing in the present application example is omitted. Hereinbelow, for the sake of concrete explanation, it is assumed that the second moving image data is a series of second procedures by the user acquired in real time. Specifically, it is assumed that the second procedure is an image including an ultrasonic probe abutting on the subject. At this time, the medical information processing apparatus 30 may be built in an ultrasonic diagnostic apparatus.

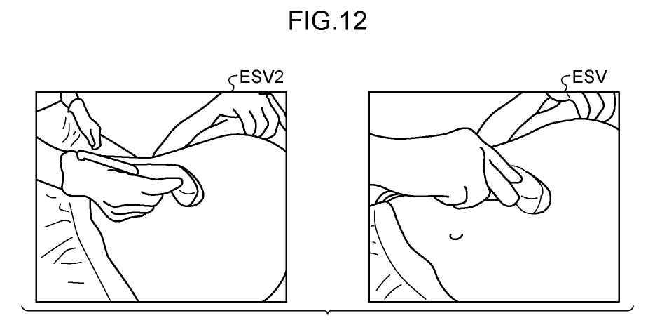

FIG. 12 is a diagram illustrating an example of an application case in the present application example. As illustrated in FIG. 12, second moving image data ESV2 is acquired in real time. At this time, the output function 34d may output the difference from the first moving image data ESV via a not-illustrated loudspeaker.

The medical information processing apparatus 30 according to the application example of the present embodiment described above, based on first moving image data obtained by capturing a series of a plurality of first procedures performed by a specific operator on a disease of a subject, generates a plurality of first texts associated with the plurality of first procedures, based on second moving image data obtained by capturing a series of a plurality of second procedures, which are normal, related to the disease, generates a plurality of second texts associated with the plurality of second procedures, extracts a difference between the first procedure and the second procedure from the first text and the second text, and outputs information regarding the difference.

As illustrated in FIG. 12, in a case where the second moving image data ESV2 is acquired in real time and the conversion-into-text and difference display processing is performed, according to the medical information processing apparatus 30 of the application example of the present embodiment, it is possible to notify the user of the difference between the expert procedure and the current procedure. Therefore, the medical information processing apparatus 30 of the application example of the present embodiment functions as a guide that causes the user to achieve the expert procedure by the conversion-into-text and difference display processing. Therefore, according to the medical information processing apparatus 30 of the application example of the present embodiment, it is possible to efficiently train a doctor in a procedure or the like in an ultrasonic diagnostic apparatus. Since other effects of the medical information processing apparatus 30 of the application example of the present embodiment are similar to those of the embodiment, the description thereof will be omitted.

In a case where the technical idea in the present embodiment is achieved by a medical information processing method (medical image processing method), the method includes, based on moving image data obtained by capturing a series of first procedures performed by a specific operator on a disease of a subject, generating a first text associated with the first procedure, based on sentence data in which information regarding a series of second procedures, which are normal, related to the disease is described, generating a second text associated with the second procedure, extracting a difference between the first procedure and the second procedure from the first text and the second text, and outputting information regarding the difference. The processing procedure of the conversion-into-text and difference display processing achieved by the medical information processing method conforms to the embodiment and the like. In addition, the effects of the medical information processing method are similar to those of the embodiment. For these reasons, the description of the processing procedure and effects of the conversion-into-text and difference display processing in the medical information processing method will be omitted.

In a case where the technical idea in the present embodiment is achieved by a medical information processing program (medical image processing program), the medical information processing program causes a computer to achieve, based on moving image data obtained by capturing a series of first procedures performed by a specific operator on a disease of a subject, generating a first text associated with the first procedure, based on sentence data in which information regarding a series of second procedures, which are normal, related to the disease is described, generating a second text associated with the second procedure, extracting a difference between the first procedure and the second procedure from the first text and the second text, and outputting information regarding the difference. For example, the conversion-into-text and difference display processing can also be achieved by installing the medical information processing program in a computer such as the medical information processing apparatus 30 illustrated in FIG. 1 and developing the program on a memory. At this time, the program that can cause the computer to execute the processing can be distributed by being stored in a storage medium such as a magnetic disk (hard disk or the like), an optical disk (CD-ROM, DVD, or the like), or a semiconductor memory.

Further, the medical information processing program may be distributed not only by the above medium, but also by using a telecommunication function such as download via the Internet. The processing procedure in the medical information processing program conforms to the conversion-into-text and difference display processing. In addition, the effects of the medical information processing program are similar to those of the embodiment. For these reasons, the description of the processing procedure and effects of the conversion-into-text and difference display processing in the medical information processing program will be omitted.

According to at least one embodiment, application example, and the like described above, it is possible to improve the training efficiency of a medical worker such as a doctor.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising:
a processing circuitry,
wherein the processing circuitry
based on moving image data obtained by capturing a series of first procedures performed by a specific operator on a disease of a subject, generates a first text corresponding to each of the first procedures,
based on sentence data in which information regarding a series of second procedures related to the disease is described, or based on moving image data obtained by capturing the second procedures, generates a second text corresponding to each of the second procedures,
extracts a difference between each of the first procedures and each of the second procedures from the first text and the second text, and
outputs information regarding the difference.

2. The medical image processing apparatus according to claim 1,
wherein the specific operator is registered as an expert regarding surgery for the disease.

3. The medical image processing apparatus according to claim 2,
wherein, to the moving image data, information regarding the specific operator is attached.

4. The medical image processing apparatus according to claim 2,
wherein the sentence data includes a guideline regarding implementation of the surgery.

5. The medical image processing apparatus according to claim 1,
wherein the processing circuitry inputs a first prompt used to generate the first text from the moving image data and the moving image data into a pre-trained generative model to generate the first text.

6. The medical image processing apparatus according to claim 1,
wherein the processing circuitry inputs a second prompt used to generate the second text from the sentence data and the sentence data into a pre-trained generative model to generate the second text.

7. The medical image processing apparatus according to claim 1,
wherein the first text and the second text each include a text for each of items including a purpose of a procedure, an operation content of the procedure, and a name of an instrument used in the procedure, and
wherein the processing circuitry extracts the difference for each of the items.

8. The medical image processing apparatus according to claim 7,
wherein, in the first text and the second text,
the processing circuitry
uses a similarity between the texts indicating the purposes to execute association according to the purposes, and
extracts the difference in the text of the operation content and the name of the instrument related to the associated purposes.

9. The medical image processing apparatus according to claim 7,
wherein, in the first text and the second text,
the processing circuitry
uses a similarity between the texts in which the purposes are combined to execute association according to the purposes, and
extracts the difference in the text of the operation content and the name of the instrument related to the associated purposes.

10. The medical image processing apparatus according to claim 1,
wherein the processing circuitry emphasizes the information regarding the difference and displays the information on a display.

11. The medical image processing apparatus according to claim 1,
wherein the processing circuitry displays on a display information regarding the first procedure not included in the sentence data as the information regarding the difference.

12. The medical image processing apparatus according to claim 8,
wherein the processing circuitry displays on a display so that the associated purposes in the first text and the second text can be visually recognized.

13. The medical image processing apparatus according to claim 8,
wherein the processing circuitry displays on a display the moving image data associated with the first procedure.

14. The medical image processing apparatus according to claim 1,
wherein the moving image data includes a series of a plurality of the first procedures performed by the specific operator,
wherein the processing circuitry generates a plurality of the first texts associated with the plurality of first procedures,
wherein the sentence data includes information regarding a series of a plurality of the second procedures and
wherein the processing circuitry generates a plurality of the second texts associated with the plurality of second procedures.

15. A medical image processing method comprising:
based on moving image data obtained by capturing a series of first procedures performed by a specific operator on a disease of a subject, generating a first text corresponding to each of the first procedures;
based on sentence data in which information regarding a series of second procedures related to the disease is described, generating a second text corresponding to each of the second procedures;
extracting a difference between each of the first procedures and each of the second procedures from the first text and the second text; and
outputting information regarding the difference.

16. A computer-readable non-volatile storage medium storing a medical image processing program causing a computer to achieve:
based on moving image data obtained by capturing a series of first procedures performed by a specific operator on a disease of a subject, generating a first text corresponding to each of the first procedures;
based on sentence data in which information regarding a series of second procedures related to the disease is described, generating a second text corresponding to each of the second procedures;
extracting a difference between each of the first procedures and each of the second procedures from the first text and the second text; and
outputting information regarding the difference.
